(19) United States
(12) Patent Application Publication
(10) Pub. No.: US 2011/0263976 A1
(43) Pub. Date: Oct. 27, 2011

(54) Methods and Devices for Endovascular Introduction of an Agent

(76) Inventors: Ali H. Hassan, Palo Alto, CA (US); Brian K. Courtney, Toronto (CA); Mark Yang, Los Gatos, CA (US); Peter J. Fitzgerald, Portola Valley, CA (US); Binh Luong, Los Gatos, CA (US); Michael Orth, Los Gatos, CA (US)

(21) Appl. No.: 13/002,641

(22) PCT Filed: Jul. 8, 2009
(86) PCT No.: PCT/US09/49966

§ 371 Date: Jun. 30, 2011

Related U.S. Application Data
(60) Provisional application No. 61/082,018, filed on Jul. 18, 2008.

(51) Int. Cl. A61M 25/14; A61B 6/00; A61M 1/00

(52) U.S. Cl. 600/433; 604/500; 604/503; 604/523

(57) ABSTRACT

Methods and devices for introduction of an agent to a vascular physiological site are provided. Aspects of the methods include employing an agent delivery device and an aspiration device to introduce and remove agent from a vascular site in a manner sufficient to minimize agent loss. Also provided are systems and kits for performing the subject methods. The subject invention finds use in a variety of different applications.

### CROSS-REFERENCE TO RELATED APPLICATIONS

Pursuant to 35 U.S.C. §119 (e), this application claims priority to the filing date of U.S. Provisional Patent Application Ser. No. 61/082,018 filed Jul. 18, 2008; the disclosure of which application is herein incorporated by reference.

### INTRODUCTION

Administration of therapeutic or diagnostic agents to a subject is typically accomplished by either localized or systemic routes. With many types of agents, localized delivery methods are desirable. For example, medical compounds of interest may have desired diagnostic or therapeutic effects within the region into which they are introduced, but also exhibit toxic or other undesirable effects when they are allowed to circulate elsewhere. In certain cases, it is desirable to introduce a higher volume of a compound to the local region than can be tolerated by other body tissues if that volume were to ultimately cause the systemic concentration to exceed a safe threshold.

A common example of such a compound is radio-opaque dye. Iodinated forms of such a dye are used routinely during catheter-based interventional procedures such as coronary, renal, neurological and peripheral arteriography.

The iodine component has a high absorption of x-rays and therefore provides a contrast medium for the radiological identification of vessels when introduced within an upstream artery. However, the use of such dyes is known to have potential toxic effects depending on the specific formulation, including direct injury to renal tubule cells, endothelial injury, bronchospasm, inflammatory reactions, pro-coagulation, anti-coagulation, vasodilation and thyrotoxicosis.

Other materials that may be introduced locally for desired effects but whose direct or other effects would be undesired elsewhere include vasoactive agents, cytotoxic agents, genetic vectors, apoptotic agents, anoxic agents (including saline), photodynamic agents, emboli-promoting particles or coils, antibodies, cytokines, immunologically targeted agents and hormones.

An important anatomic concept with respect to the vasculature and other conduits supplying and draining an organ is the principle that a tissue or organ and regions of the organ have a limited number of primary supply conduits and a limited number of draining conduits. Material introduced into the upstream side of the target tissue will typically be dispersed among the diverging arterioles and capillaries, which then converge into a collection of common venules and vein (s) downstream, e.g., in a physiological efferent fluid collection site. For example, the myocardium of the heart is fed by the right coronary, left anterior descending and left circumflex arteries. Each of these arteries enters a capillary network that eventually converges into the small and middle cardiac vein, anterior interventricular vein and posterior vein of the left ventricle. These veins are all tributaries of the coronary sinus, which may be viewed as a cardiovascular efferent fluid collection site. Material introduced into any of the aforementioned coronary arteries that travels through the capillary network will enter the coronary sinus providing an opportunity to collect it before it returns to the systemic circulation. In another example, the brain is fed by the carotid and vertebral arteries which enter a highly anastomotic network. Blood flow through the brain substantially drains to the systemic circulation via a network of sinuses that converge onto the internal jugular veins. In yet another example, each kidney is substantially supplied by a renal artery and drained by a renal vein. In yet another example, a tumor or metastatic lymph node may have a set of primary afferent (supply) conduits and a set of primary efferent (drainage) conduits. In yet another example, the lungs are supplied by a pulmonary artery and its branches, and are drained by the pulmonary veins and their tributaries into the left atrium.

### SUMMARY

Methods and devices for vascular introduction of an agent are provided. Aspects of the methods include first positioning a distal end of an agent delivery device to a vascular location upstream of a vascular physiological site; and an aspiration device at least proximal to the physiological site. Next, agent is introduced via the agent delivery device to the vascular location upstream of the physiological site and the aspiration device is activated when the agent is at least predicted to be present in the physiological site to remove fluid comprising the agent from the subject. Aspects of the methods include introducing the agent and activating the aspiration device in a manner sufficient to minimize agent loss, e.g., via unintended leakage into the general circulation. Also provided are systems and kits for performing the subject methods. The subject invention finds use in a variety of different applications.

### BRIEF DESCRIPTION OF THE FIGURES

FIGS. 1A and 1B provide a view of an injection device and aspiration element being employed to introduce and remove an agent from an organ according to an embodiment of the invention.

FIG. 2 provides a view of an injection device that includes a non-occlusive flow focusing element according to an embodiment of the invention.

FIGS. 3A and 3B provide a view of an injection device that includes a non-occlusive flow focusing element according to another embodiment of the invention.

FIG. 4 provides a view of an injection device that includes an occlusive flow focusing element according to an embodiment of the invention.

FIGS. 5A and 5B provide views of an injection device that includes an occlusive flow focusing element according to an embodiment of the invention.

FIGS. 6A and 6C provide views of an injection device that includes an occlusive flow focusing element according to an embodiment of the invention.

FIG. 7 provides a view of an injection device that includes a distal end detector, e.g., for determining hemodynamic signatures, according to an embodiment of the invention.

FIGS. 8 and 9 provide schematic representations of system according to two different embodiments of the invention.

### DETAILED DESCRIPTION

Methods and devices for vascular introduction of an agent are provided. Aspects of the methods include first positioning a distal end of an agent delivery device to a vascular location upstream of a vascular physiological site; and an aspiration device at least proximal to the vascular physiological site. Next, agent is introduced via the agent delivery device to the vascular location upstream of the vascular physiological site and the aspiration device is activated when the agent is at least predicted to be present in the vascular physiological site to remove fluid comprising the agent from the subject. Aspects of the methods include introducing the agent and activating the aspiration device in a manner sufficient to minimize agent loss, e.g., via unintended leakage into the general circulation of the subject. Also provided are systems and kits for performing the subject methods. The subject invention finds use in a variety of different applications.

Before the present invention is further described, it is to be understood that this invention is not limited to particular embodiments described, as such may vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Methods recited herein may be carried out in any order of the recited events which is logically possible, as well as the recited order of events.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Methods and Devices

Aspects of the invention include methods for delivering an agent to and removing an agent from a vascular physiological site of a living subject. The vascular physiological site may be in a vessel, and in such embodiments the methods are methods of endovascularly delivering and removing an agent from a living subject. The living subject is generally an animal, where in certain embodiments the animal is a “mammal” or “mammalian.” The terms mammal and mammalian are used broadly to describe organisms which are within the class mammalia, including the orders carnivore (e.g., dogs and cats), rodentia (e.g., mice, guinea pigs, and rats), lagomorpha (e.g. rabbits) and primates (e.g., humans, chimpanzees, and monkeys). In certain embodiments, the subjects (i.e., patients) are humans.

Methods of the invention include positioning a distal end of an agent delivery device to a vascular location upstream of the vascular physiological site. In certain embodiments, the vascular physiological site is a vascular efferent fluid collection site, where fluid from at least two different vessels joins into a single vessel. In certain embodiments, the vascular physiological site is a cardiac site, e.g., a cardiovascular fluid collection site, where fluid from at least two different veins joins into a single venous structure. In one embodiment of interest, the cardiovascular efferent fluid collection site is the coronary sinus. In yet other embodiments, the efferent fluid collection site may be an artificially, e.g., surgically, produced, fluid collection site, e.g., a non-naturally occurring fluid collection site produced by surgically joining two or more vessels together, etc.

The agent delivery device of embodiments of the methods is a catheter device that includes a proximal end, a distal end and at least one lumen that opens at the distal end. During use, the proximal end of the agent delivery device is operably coupled to a source of agent, e.g., a fluid source of agent (such as a reservoir), and an injection mechanism that operates to introduce the agent from the lumen of the delivery device at the distal end of the device into the injection site. For example, the device may include a pressure mechanism that operates to force liquid under pressure through the lumen of the device and out the distal end into the vascular location upstream of the vascular physiological site of interest. In some embodiments, the agent delivery device may include one or more additional elements at the distal end, such as an agent delivery focusing element, a support structure, a sensor or detector, etc., as discussed further below.

By “upstream” is meant that the vascular location into which the agent is delivered is located at a position upstream of the vascular physiological site with respect to the direction of majority of fluid flow through the vascular physiological site, i.e., the direction that most of the fluid, if not all of the fluid, flows through the vascular physiological site. The distance between the site of agent delivery location and the vascular physiological site may vary depending on the particular application. In certain embodiments, this distance ranges from 1 mm to 100 cm, such as from 1 mm to 10 cm and including from 1 mm to 1 cm. In certain embodiments, the upstream vascular location is an arterial (or pre-arteriolar) location and the vascular physiological site is a venous location.

In addition to positioning a distal end of an agent delivery device into an upstream vascular location, the methods also include placing a distal end of an aspiration device at least proximal to the vascular physiological site, as described in greater detail below. Accordingly, in practicing embodiments of the subject methods, a fluid removal device, e.g., an aspiration device, is introduced into (i.e., positioned at), a target site. The target site is at least proximal to the physiological site, which may be an efferent fluid collection site and for ease of illustration is now further described as an efferent fluid collection site. By “at least proximal to” is meant that the target site is either upstream or downstream of the collection site, or the same as the collection site, so long as placement of the aspiration element at the target site provides for the desired removal of agent from the collection site upon actuation of the aspiration element, as described in greater detail below. In certain embodiments, the target fluid removal site is at a distance of 40 mm or less from the efferent fluid collection site, e.g., at a distance of 15 mm or less from the efferent fluid collection site.

Following positioning of the agent delivery and aspiration devices, agent is introduced via the agent delivery device to the vascular location upstream of the physiological site and the aspiration device is activated when the agent is at least predicted to be present in the physiological site to remove fluid comprising the agent from the subject. The rate of agent fluid introduction may vary. In certain embodiments, the rate of agent fluid introduction ranges from 100 ml/sec to 0.1 ml/sec, such as from 10 ml/sec to 0.2 ml/sec and including from 3 ml/sec to 0.5 ml/sec. Injection of agent may be continuous, or in certain embodiments pulsed, e.g., in accordance with hemodynamic profiles, as described in greater detail below.

Following positioning of the aspiration element and flow modulator at the target site, the aspiration device is activated when the agent to be removed is at least predicted to be present in the target site. Activation occurs in a manner effective to remove fluid comprising the target agent from the subject. Embodiments are characterized in that the agent is selectively removed from the efferent fluid collection site. As reviewed above, aspects of the invention include the selective removal of agent from the efferent fluid collection site. By “selectively removal” is meant that the subject methods remove fluid from the target site in a manner that selectively or preferentially removes fluid that is at least predicted to include the agent, where the removed fluid is not returned to the body, at least not without processing to remove the target agent present therein. In certain embodiments the removed fluid is simply disposed of, such that the methods include a step of disposing of the removed fluid, while in other embodiments the fluid is processed (e.g., filtered) and then returned to the subject, as reviewed in greater detail below. Depending on the particular protocol and device employed, as described in greater detail below, the fluid may be continuously collected at the fluid collection site but not removed from the body unless it is at least predicted to include agent, e.g., as occurs in those embodiments where fluid is collected at the fluid collection site but immediately shunted back to the subject if it is not at least predicted to include agent. By “at least predicted” is meant that the bulk or majority of the fluid removed from the site is fluid that is either anticipated to include the agent, e.g., fluid in which the presence of the agent is inferred, or fluid that is known to include the agent, e.g., fluid in which the presence of the agent is detected. Depending upon the particular embodiment of the invention being practiced, in selectively removing fluid from the target fluid collection site and subject, fluid may be removed from the site and subject for a period of time which commences prior to when agent is at least predicted to be in the site, and extend for a period of time after agent is at least predicted to be in the site. In such embodiments, the period of time during which fluid is collected before and/or after agent is at least predicted to be in the site is a fraction or portion of the total period of time during which agent is predicted to be at the fluid collection site, such as less than 50%, such as less than 25% including less than 10-15% of the total time period during which agent is predicted to be at the fluid collection site.

In certain embodiments, the subject methods do not remove all fluid from a target and efferent fluid collection site, but just fluid that is at least predicted to include the target agent of interest. In other words, in practicing the subject methods, not all fluid from an efferent fluid collection site present over a given period of time is removed, only fluid that is at least predicted to include the target agent of interest that is to be removed. Put another way, over a given period of time where fluid that does and does not include the target agent flows through the efferent fluid collection site and/or a target fluid collection site, only fluid that is at least predicted, e.g., is anticipated or known to include the agent, is removed from the site and subject, while fluid that does not likely include the target agent is preferentially not removed from the site and subject.

Another aspect of certain embodiments of the subject methods is that, in certain embodiments, not all of the agent that is administered prior to practice of the subject methods is removed from the subject. In other words, only a portion of the administered agent is removed from the living subject by the methods of the these embodiments. By portion is meant 20% or more, such as 50% or more and including 70% or more of the administered agent is removed by the subject methods, where in certain embodiments, the portion removed is 75% or more, 80% or more, and even 90% or more. However, as not all of the agent is collected during practice of embodiments of the subject methods, in certain embodiments 1% or more of the originally administered agent remains in the subject or patient, such as 5% or more or even 10% or more.

Agent is selectively removed from the target site, which may or may not be the efferent fluid collection site, according to the subject methods by removing, e.g., aspirating, fluid from the target site and subject, substantially only when the agent is at least predicted to be present in the target site, as described above. As such, when agent is at least predicted to be present in the target site, fluid is removed from the site and subject. Conversely, in certain embodiments when agent is not predicted to be present in the site, fluid is not removed at least from the subject, and in certain embodiments not from the site. Accordingly, in certain embodiments, upon detection of agent or in anticipation of the presence of agent in the fluid collection site, fluid is removed or aspirated from the site and subject. In other embodiments, when the target agent is not detected or anticipated to be present in the site, fluid is not removed from the site, with the exception of a short period of time before and/or after the time when agent is at least predicted to be in the target site, as described above.

In certain embodiments, fluid is selectively removed by actuating a fluid removal element, e.g., aspiration device, such as the devices described below, for a defined period of time following administration of the agent to the subject, e.g., an absolute preset period of time, a period of time as defined by a physiological metric, e.g., heart beat, etc.

An aspect of the invention is that both the introducing of the agent and activating the aspiration device are performed in a manner sufficient to minimize agent loss. As such, agent introduction by the introduction device is performed in a manner sufficient to reduce, if not eliminate, agent loss at the site of introduction. Agent loss refers to agent that is delivered to the site and not recovered from the site. In other words, agent is considered lost if it has been delivered by the delivery device and not recovered by the aspiration mechanism. Accordingly, aspects of the invention reduce bodily agent load by means of minimizing agent loss (spillage) at injection site, combined with means for agent capture from an efferent vessel. As the methods and systems of invention minimize loss of agent, i.e., agent that is not ultimately collected by the aspiration device, only insubstantial amounts of agent, if any, are delivered to locations of the body that are not located somewhere between the upstream vascular location (i.e., injection site) and the vascular physiological site (i.e., collection site). By insubstantial amount is meant 50% or less, such as 20% or less, including 10% or less. Where desired, methods of the invention may include use of agent delivery and removal module (e.g., as described in greater detail below) to coordinate delivery and removal in a manner sufficient to minimized agent loss.

In certain embodiments, agent loss is minimized by introducing the agent in a manner that includes determining fluid flow parameters (e.g., hemodynamic parameters) at the site of introduction and introducing the agent at a time and/or rate selected in view of the determined flow parameters. By fluid flow parameters is meant parameters that impact or affect the flow of fluid through the injection site, where such parameters include, but are not limited to, rate of fluid flow, direction of fluid flow, local endovascular pressure, etc. The flow parameters may be hemodynamic (vital) parameters in a target vessel, vital patterns in a target organ, etc.

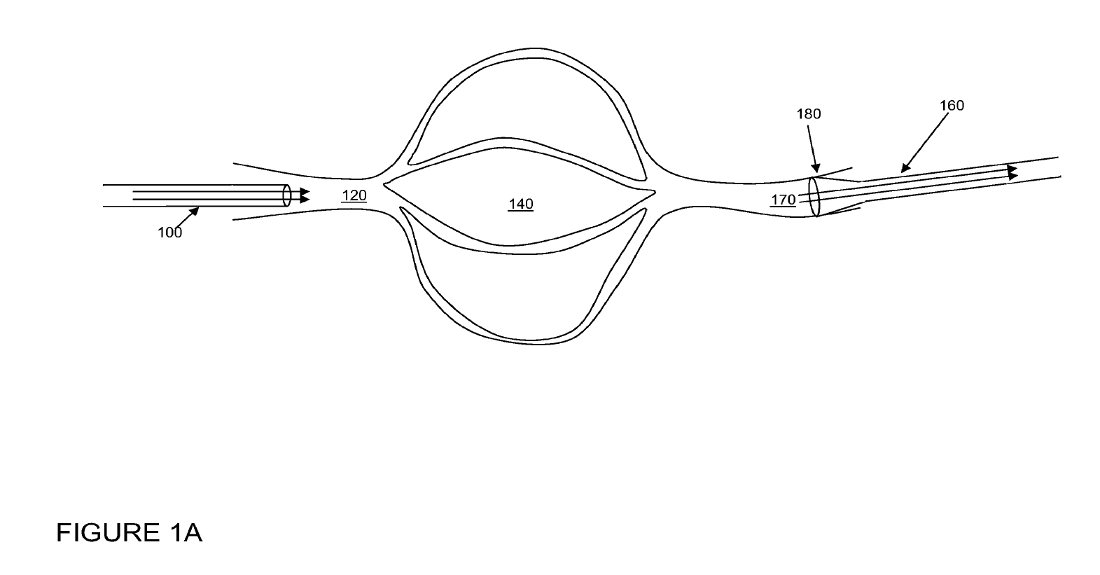

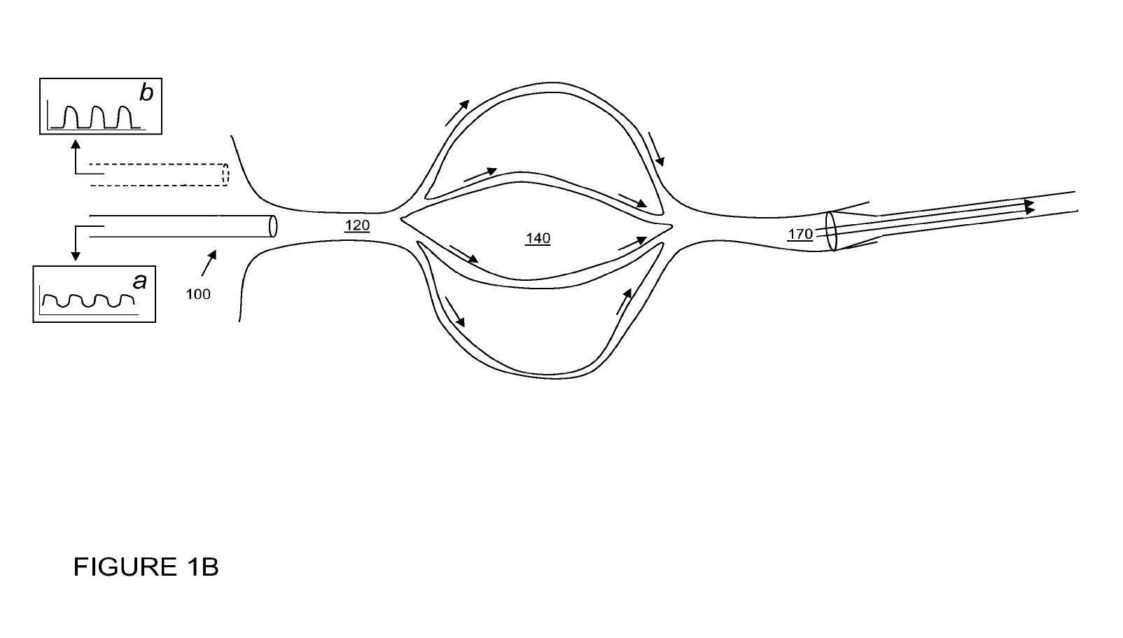

FIG. 1A shows a view of this embodiment of the invention. In FIG. 1A, a non-occlusive injection catheter 100 is positioned into an afferent (arterial) vessel 120 of an organ 140, e.g., the heart. Agent is introduced into the injection site in a manner such that it is introduced at a time when fluid is flowing at a maximum rate in the direction of the vascular physiological site. For example, agent can be introduced into the injection site at a time at which fluid is flowing through site 120 at the fastest rate, e.g., during diastole of the cardiac cycle. The rate of fluid delivery may also be chosen so as not to overwhelm the amount of fluid that can be accommodated by the afferent location 120 without leakage of the agent in a direction upstream from afferent location 120. In this manner, agent is introduced to the afferent site 120 at a time and/or rate selected in view of the determined flow parameters of site 120 and/or organ 140. An embodiment of this approach is further illustrated in FIG. 1B. In FIG. 1B, injection catheter 100 is positioned into afferent (arterial) vessel 120 of an organ, e.g., as described in connection with FIG. 1A above. The catheter tip is oriented such that a hemodynamic signature of the target injection site can be recognized, e.g., as illustrated by plots “a” and “b”. Agent is injected into the site following a biological pattern such as hemodynamic (vital) parameters in target vessel, e.g., at desired points of the hemodynamic signature. In certain embodiments, rate of fluid injection may be determined from the dimensions of the injection device or one or more components thereof, and matched to a hemodynamic signature, as desired. In certain embodiments, the injection device is configured to provide for heat exchange with the fluid, e.g., when the temperature of the agent is matched to the temperature of the target injection site, and in special cases of cold/heat delivery conditions.

Also shown in FIG. 1A is aspiration device 160. Aspiration devices of interest include, but are not limited to, those described in U.S. Pat. Nos. 7,300,429 and 7,211,073, the disclosures of which are herein incorporated by reference. In certain embodiments, aspiration devices that include a flow modulator at their distal ends are employed. In such devices, the flow modulator 180 is configured to converge fluid flow paths, which may be parallel or intersecting, into the aspiration element. By intersecting flow paths is meant flow paths that are not parallel, where the non-parallel flow paths may or may not intersect each other at an angle, e.g., a right angle. In certain embodiments, the intersecting flow paths are the product of two or more different tributary vessels into the efferent fluid collection site. Aspiration devices that include a flow modulator are further described in U.S. patent application Ser. No. 12/138,291, the disclosure of which is herein incorporated by reference. An example of such an embodiment is shown in FIG. 1A, wherein aspiration element or device 160 includes a flow modulator 180 at its distal end. As depicted in FIG. 1A, aspiration of fluid from efferent vessel using a flow modulator occurs when agent is detected or at least predicted to be present in the efferent vascular physiological site 170.

In certain embodiments, aspiration of fluid occurs in response to the detection of the presence of agent at efferent site 17, e.g., in response to a signal obtained from a detector. Accordingly, in certain embodiments, the methods include a detection step, where a procedure relevant parameter is detected using an appropriate sensor (i.e., detector) element. A variety of different procedure relevant parameters may be detected, as desired. Such parameters include, but are not limited to: the agent itself (which may be detected both directly and/or indirectly), flow dynamic, e.g., hemodynamic parameters, anatomical parameters, etc. A variety of sensors may be employed, including but not limited to: impedance sensors, ultrasound sensors, Doppler sensors, optical sensors, temperature sensors, binding-event sensors, etc.

In certain embodiments, the methods include a step of detecting one or more fluid flow parameters, such as hemodynamic parameters. For example, fluid flow may be assessed at various locations of an efferent fluid collection site during a given protocol. With respect to the coronary sinus, fluid flow may, for example, be assessed at the flow outlet of the flow modulator to the aspiration element (e.g., to provide activation efficiency of aspiration) and/or at the ostium (OS) of the coronary sinus (e.g., for assessing reflux from the right atrium). A variety of different types of flow dynamic sensors may be employed, as desired, where such sensors include deflection sensors, thermal sensors, sensors of oxygen contents e.g., that provide an assessment of flow direction or orifice of a tributary during device insertion, etc.

In certain embodiments, the methods include a step of detecting the presence of target agent in the site and then removing fluid, and agent present therein, from the site in response to detection of the presence of target agent in the site. The presence of the agent may be detected directly or indirectly. In some embodiments, when agent is no longer detected in the efferent fluid collection site, fluid is no longer removed from the site. Thus, fluid is only removed from the efferent fluid collection site and subject over a time period that substantially overlaps the period in which the target agent is present in the efferent fluid collection site.

In practicing these embodiments of the subject methods, the agent may be detected in the fluid collection site using a number of different protocols. In certain embodiments, agent is visually detected by a skilled operator, who then removes fluid in response to visualizing agent, e.g., according to the protocols described below, present in the fluid collection site. In yet other embodiments, agent detection devices that are operatively connected to a fluid removal device are employed, where a signal from the detector that agent is present in the fluid collection site automatically actuates a fluid removal device, e.g., aspiration unit.

In certain embodiments, the system includes a detector (i.e., sensor) component, e.g., for detecting the agent of interest (or a proxy therefore). The agent of interest may be detected using a number of different approaches. In certain embodiments, properties of the agent itself are detected. For example, specific binding of the agent may be employed, e.g., using a binding event sensor; optical/photometric approaches for detecting the agent, e.g., reflectance, transmission, evanescence, etc., may be employed; physical, e.g., viscosity, changes caused by the agent may be employed; electrical, e.g., conductivity, changes caused by the agent may be employed; radioactive, e.g., radiosorbance, approaches may be employed; fluorescence changes caused by the agent may be employed; acoustic, e.g., ultrasonic: echogenicity, scattering, etc. changes caused by the agent may be employed, etc.

In certain embodiments, changes in the fluid caused by the presence of the agent are employed to detect the presence of the agent. Changes of interest in a given fluid include, but are not limited to: changes in number of blood cells per volume; changes in optical properties; changes in chemical properties; changes in physical properties (density, hematocrit, viscosity, temperature); changes in hemodynamic properties (velocity); changes in overall imaging properties of blood (ultrasonic, radioactive, radiosorbent, fluorescent, etc.).

To aid in the detection of the agent, in certain embodiments the agent will be one that is labeled with a detectable label, e.g., agent that is has been labeled with a detectable label prior to its introduction into the patient. The agent may be directly labeled with the detectable label, or associated with a detectable label such that the agent is indirectly detectable in that detection of the label also indicates the presence of agent which is presumed or inferred to be within the vicinity of the label. The nature of the label may vary, and may be a radio label, fluorescent label, chromogenic label (e.g., that has a pigment detectable in the optically visible spectrum), etc.

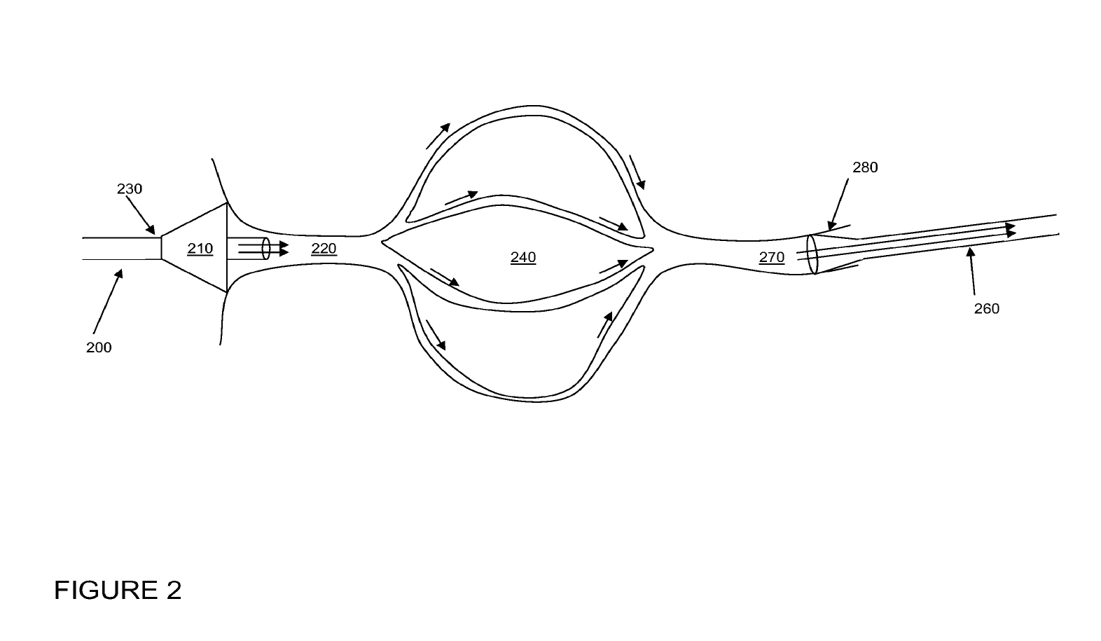

In certain embodiments, the agent delivery device includes an agent delivery focusing element, where the focusing element may be an occlusive element or a non-occlusive element. FIG. 2 provides a view of an embodiment in which the agent delivery device 200 (in the form of an injection catheter) includes an agent delivery focusing element at the distal end of the agent delivery device in the form of a non-occlusive flow modulator 210. In FIG. 2, the non-occlusive injection catheter is positioned into afferent (arterial) vessel site 220 of an organ 240, e.g., a coronary artery of the heart. The flow modulator 210 is configured to substantially if not completely isolate the vessel lumen upstream of injection site 220, at least during agent introduction, e.g., so as to prevent agent from entering vessel regions upstream of the introduction site. The flow modulator is non-occlusive, such that lumen isolation occurs in a non-occlusive manner. By non-occlusive manner is meant that the lumen of the vessel is not blocked when agent is not being delivered to the afferent site 220. In certain embodiments, the flow modulator provides a communication zone between two fluid compartments. The communication zone allows for maintaining endovascular pressure at physiological levels throughout the hemodynamic cycle. In certain embodiments, the non-occlusive catheter includes a communication port 230 proximal to the flow modulator 210 which allows for fluid flow into the flow modulator 210 at region 230 when agent is not being delivered to site 220. The communication port also allows for maintaining endovascular pressure at physiological levels, in order to maintain a reasonably functional perfusion pressure to the target tissue. In certain embodiments, lumen isolation with flow modulator 210 is continuous during operation agent delivery. In certain embodiments, lumen isolation can follow biological patterns in the target vessel/organ, e.g., flow parameters, as described above. As with the aspiration element, the flow modulator of the delivery device, when present, may vary. Of interest are flow modulators such as described in a flow modulator are further described in U.S. patent application Ser. No. 12/138,291, the disclosure of which is herein incorporated by reference. The system depicted in FIG. 2 includes aspiration element 260 with flow modulator 280 which removes fluid containing agent from efferent site 270 in a manner analogous to that described above in connection with the description of FIG. 1A.

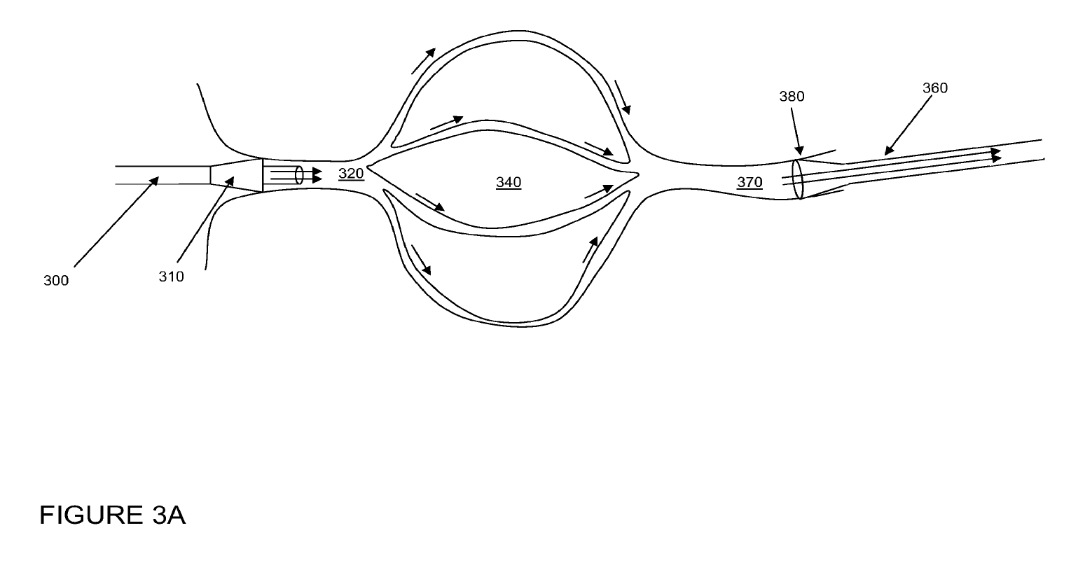

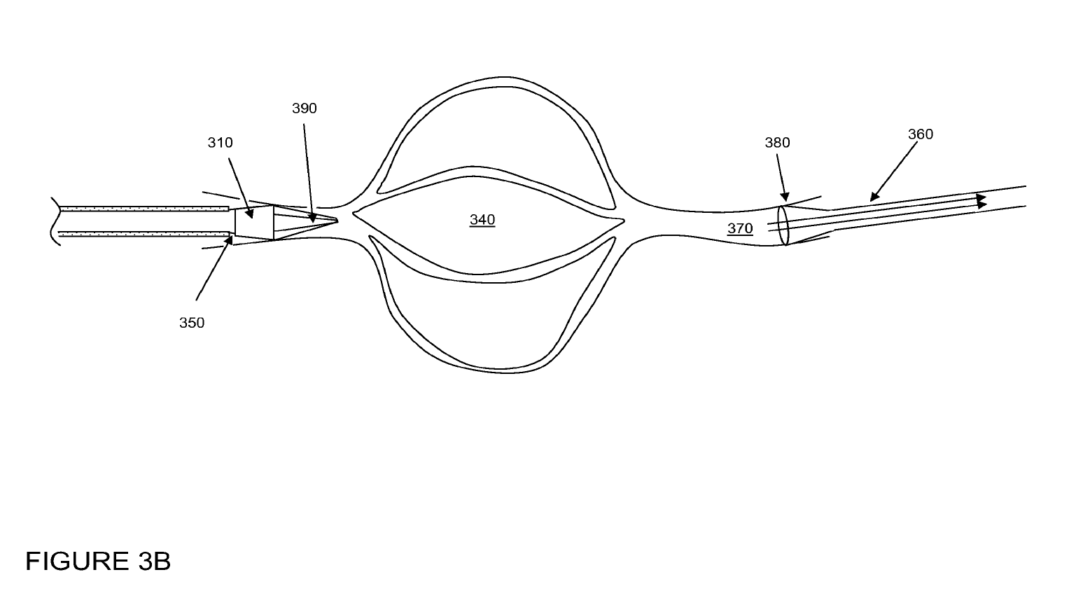

FIG. 3A provides a depiction of a variation of the system and method shown in FIG. 2. In the embodiment depicted in FIG. 3A, flow modulator 310 of agent delivery device 300 is configured to be positioned in the afferent site 320 during introduction of agent. The flow modulator is configured to alter fluid flow into afferent site 320 by interacting with fluid flow at a region upstream of site 320. In certain embodiments, the flow modulator 310 is configured to antagonize reverse flow through afferent site 320. The flow modulator 310 is again a non-occlusive flow modulator, such that when agent is not being delivered to site 320, the vessel is not occluded by the flow modulator. The non-occlusive nature of the flow modulator may be provided in a number of different ways (e.g., a communication port or zone, valve, flap, etc.), including those described in connection with the embodiment of the system shown in FIG. 2, as described above. Also shown in this embodiment are organ 340 and efferent vascular physiological site 370.

FIG. 3B depicts another embodiment of a flow modulator 310 as shown in FIG. 3A which includes a communication zone 350 for free blood flow. The communication zone 350 allows for maintaining endovascular pressure at physiological levels. Also shown in the device depicted in FIG. 3B is a support structure 390 which maintains the positioning and configuration of the flow modulator at the desired afferent site. A support structure can be configured to allow the flow of blood and/or agent through the structure, and can be configured to be collapsible or retractable within the agent delivery device, as desired. The support structure can have a cone-shaped configuration as shown in FIG. 3B, however the structure can also have any other suitable configuration such as an umbrella shape, cylindrical shape, or have prongs, etc. In some embodiments, a support structure can also be used with an aspiration device, to maintain the positioning and configuration of the flow modulator at the desired efferent site.

The system depicted in FIGS. 3A and 3B includes an aspiration element 360 with a flow modulator 380 that removes fluid containing agent from an efferent fluid collection site in a manner analogous to that described above in connection with the description of FIG. 1A.

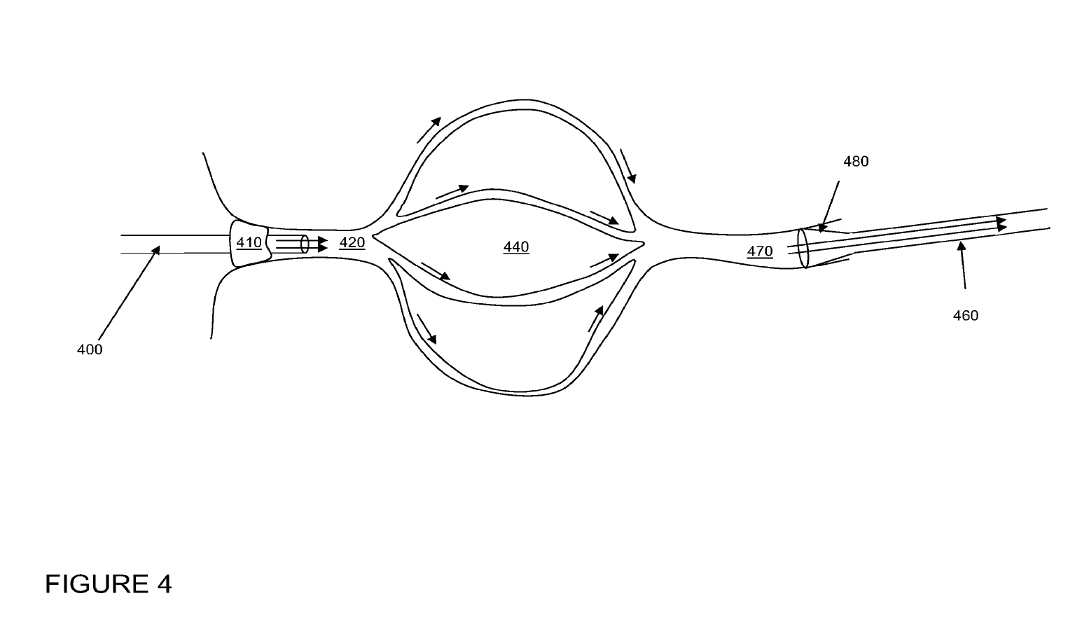

In certain embodiments, the agent delivery focusing element is an occlusive element. FIG. 4 shows a depiction of an embodiment of such a device where an occlusive injection catheter 400 is positioned into afferent (arterial) vessel site 420 of an organ 440. The injection catheter 400 has an occlusion element 410 which is configured to substantially, if not completely, occlude the vessel lumen upstream of injection site 420. The occlusion element may be any of a variety of different structures, including a balloon or analogous structure which provides for occlusion of the lumen. See e.g., U.S. Pat. Nos. 6,554,819 and 7,363,072 for disclosures of vascular occlusive elements of interest, the disclosures of which are herein incorporated by reference. The system depicted in FIG. 4 also includes aspiration element 460 with flow modulator 480 which removes fluid containing agent from efferent site 470 in a manner analogous to that described above in connection with the description of FIG. 1A.

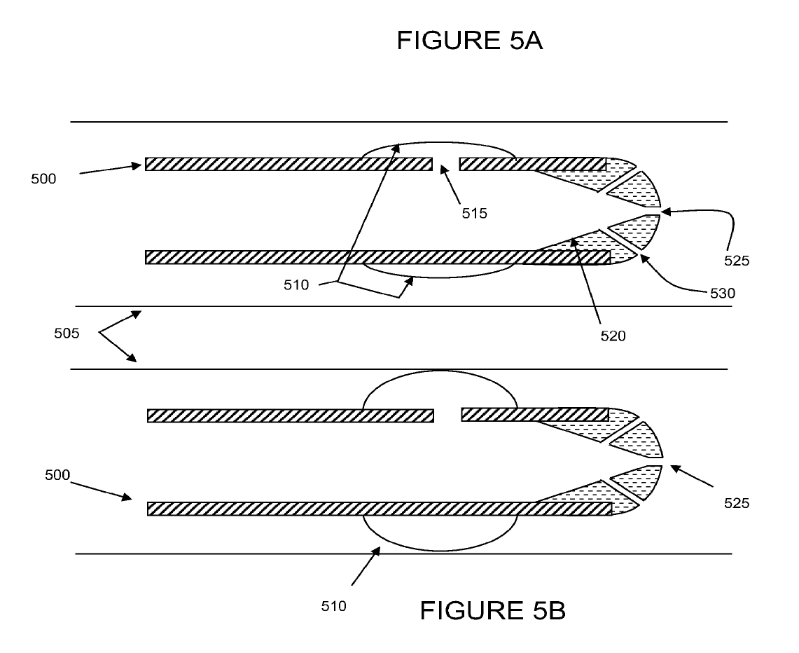

In certain embodiments, occlusion and injection are timed to start/terminate simultaneously. An example of such an embodiment is depicted in FIGS. 5A and 5B. In this embodiment, a catheter 500 positioned in blood vessel 505 is shown with a special distal tip configured with a central orifice 525 and at least one spray port 530. During injection through the at least one spray port 530, the pressure in the distal portion of the inner lumen of the catheter builds up and causes the compliant balloon 510 to expand, preventing flash back (i.e., leakage of the agent in a direction upstream of the injection). Once the injection is done, the pressure inside the distal portion of the inner lumen decreases and the balloon deflates. In this embodiment, the tip of the catheter has one or more (where several are depicted) small spray ports that create some resistance to fluid flow out of the catheter, thus causing the pressure in the distal portion of the inner lumen of the catheter to build up during injections. The spray ports are also configured to allow the built up pressure to reduce when the injection is near completion. The number and size of holes determines the amount of resistance, and can thereby determine the amount of pressure generated inside the distal end of the catheter and the balloon. Alternatively, at least some of the holes can be replaced by a pressure relief mechanism (e.g., a valve) that opens at a pre-selected pressure. In some instances, one or two small holes may be present to provide a relief to allow for balloon deflation. This embodiment provides for the production of a temporary occlusion which prevents back flushing, that automatically deflates after the injection is done. In this embodiment, the catheter includes wire port 525 and a wire-directing guide in the tip 520 that directs the wire through wire port 525. In this embodiment, the wire-directing guide in the tip of the catheter which directs the wire through the wire port is the angled inner walls of the distal catheter, which direct the wire to wire port 525. In other embodiments, the elements of a wire port and a guide which directs the wire through the port can be replaced by having a separate wire lumen if desired. Also shown is balloon inflation port 515.

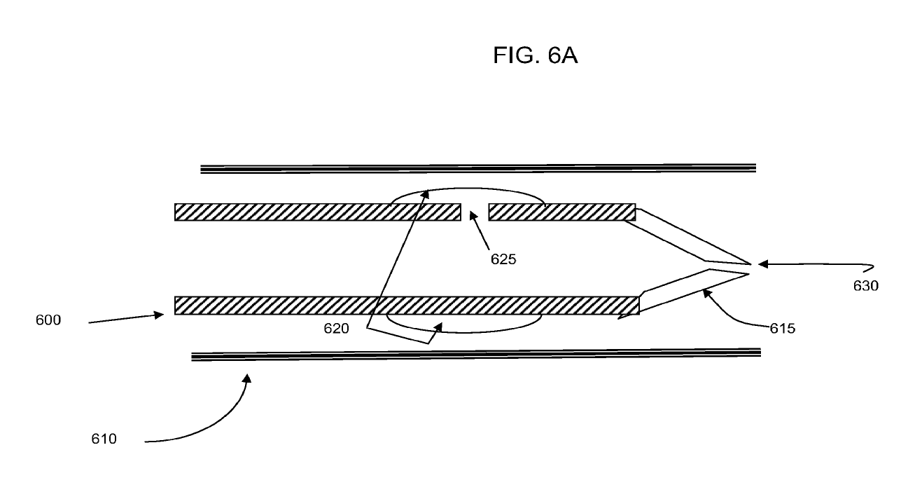

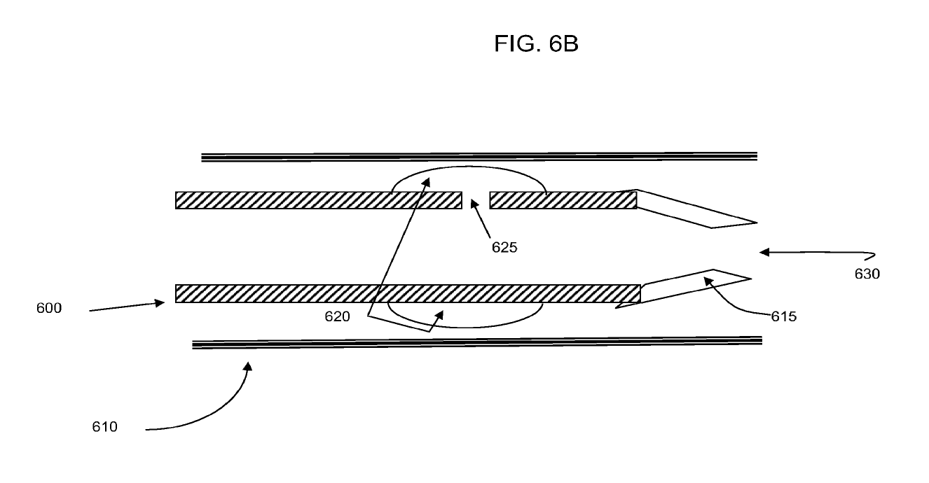

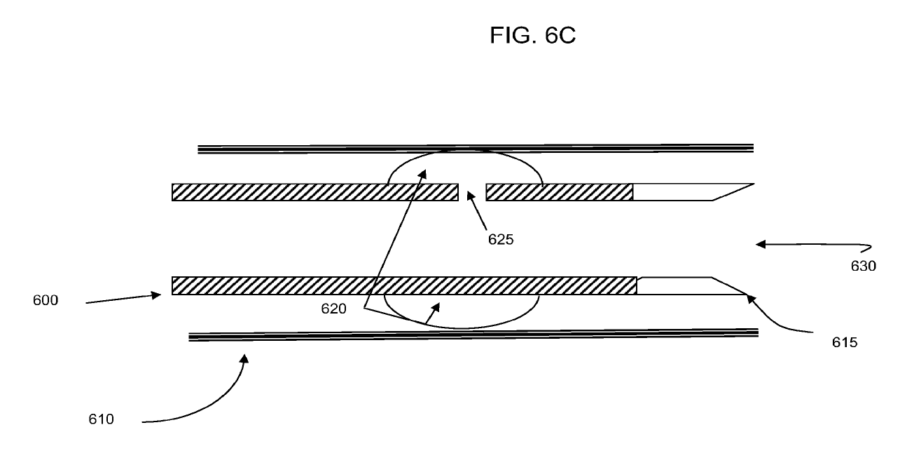

As shown in FIGS. 6A to 6C, in another embodiment of the fluid delivery device, a catheter 600 can have a pressure responsive (such as a soft, deformable) distal tip 615 that initially restricts flow under first pressure conditions (such as low pressure conditions when no fluid is flowing out the catheter). In FIG. 6A, catheter 600 is shown in blood vessel 610. As no fluid is flowing through catheter 600, tip 615 assumes a configuration in which opening 630 is at a minimal diameter. As fluid flow increases through the catheter, pressure builds up (see FIG. 6B) to second pressure conditions, the deformable tip 615 opens up in response to the increase in pressure to the second pressure conditions (thereby increasing the diameter of opening 630), and the occlusion balloon 620 inflates as fluid flows into the occlusion balloon 620 via port 625. Upon maximum fluid flow through catheter 610 and attainment of third pressure, pressure responsive deformable distal tip 615 assumes a position of maximal opening such that the diameter of opening 630 is at a maximum and balloon 620 is at maximal inflation such that it touches the lumen of vessel 610, as shown in FIG. 6C. As the injection of agent comes to an end and pressure in the catheter 600 is reduced to the first pressure, the catheter tip 615 and balloon transition from the state of maximum opening and inflation as shown in FIG. 6C back to the state of minimal opening and deflation as shown in FIG. 6A. For ease of description, the various states illustrated in FIGS. 6A to 6C have been described in terms of first, second and third pressures and configurations. However, as flow varies the pressure may continuously change and the configurations of the tip and balloon may correspondingly continuously change.

In yet other embodiments, the occlusion is timed to be of a duration that is shorter than the injection, e.g., 50% shorter, including 10% shorter. As desired, occlusion can follow biological patterns in target vessel/organ, e.g., as described above. In certain embodiments, the methods include identification of a desired target injection site based on a detected hemodynamic signature(s) of the target organ/tissue. In such embodiments, the injection device is configured to detect or identify a hemodynamic signature at its distal end and inject when a desired portion of the signature is reached. An example of such an embodiment is depicted in FIG. 1B. In FIG. 1B, the distal end or tip of the injection catheter is positioned such that the hemodynamic (vital) parameter (depicted in the FIG. 1B as plots “a” and “b”) of target injection site is recognized. If the catheter tip is located outside the desired injection site, e.g., the detected hemodynamic signature (plot “b”) is different from the signature observed at the desired location (plot “a”). Hemodynamic signature outputs to the operator, e.g., the form of plots “a” and “b” (See FIG. 1B), may be employed by the operator to ensure that desired positioning of the detector is achieved.

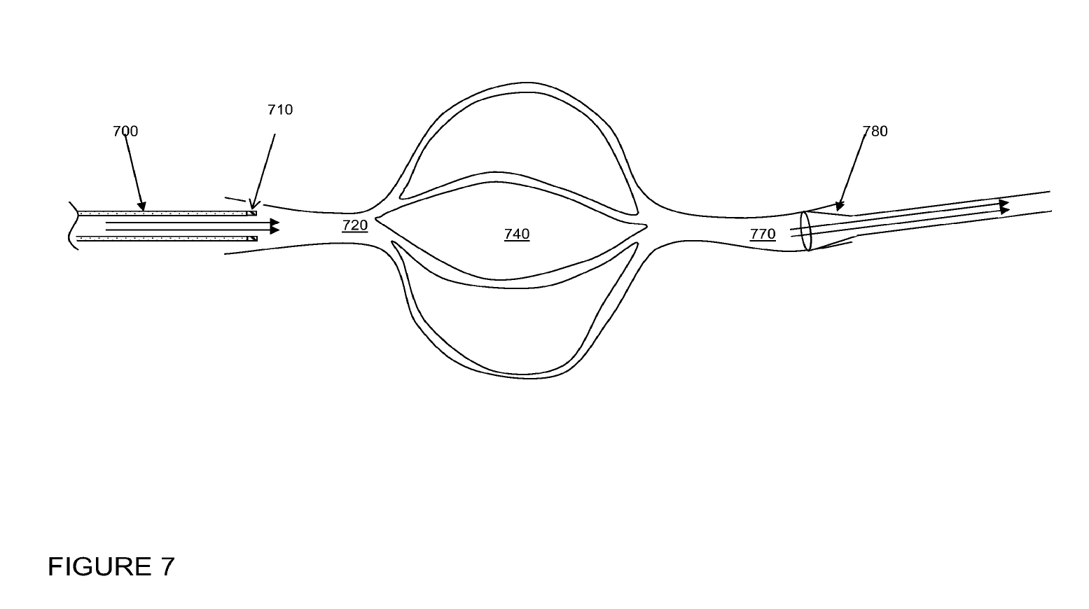

Determining a desired hemodynamic curve can be achieved using the same injection catheter as shown in FIG. 1A. In one embodiment, the fluid column inside the injection catheter is employed to translate pressure waves, and thus provide pressure tracings, that reflect the hemodynamics at the target injection. In another embodiment, the agent delivery device, e.g., injection catheter, includes a sensor (i.e., detector). An appropriate sensor 710, e.g., at the distal end of the catheter 700 as shown in FIG. 7, can be employed to map the flow pattern in target injection site, in order to provide hemodynamic patterns which can be used for injection. One or more sensors or detectors can be used, and can include for example, a flow sensor and/or a pressure sensor. Where desired, the obtained hemodynamic signature can be matched against signatures of known sites, e.g., appropriate references or controls, in order to verify correct positioning of the injection catheter tip. Also shown in FIG. 7 are afferent site 710, organ 750, efferent site 770 and fluid collector 780.

In certain embodiments, the method includes determining an agent delivery efficiency value and outputting that determined value to a user. An example of such an efficiency value is a value that represents how much delivered agent, if any, was not recovered by the aspiration device. In certain embodiments, the agent delivery efficiency value is determined by comparing the quantity of agent introduced to the vascular location upstream of the vascular physiological site and the amount of agent removed from the vascular physiological site, i.e., the afferent and efferent sites respectively as depicted in FIGS. 1A to 7.

In certain embodiments, the methods include a step of detecting anatomical structures or features, e.g., to assist in proper placement of the devices, e.g., the injection and/or aspiration devices, at the desired vascular sites. For example, detectors for assessment of branching points of tributaries of interest (e.g., middle cardiac vein, posterior vein of left ventricle, lateral vein of left ventricle, and other vascular tributaries) may be employed (e.g., to aid in axial positioning of the device at the target site). Examples of such detectors include flex detectors (e.g., as described in United States Published Application No. US-2006-0173365-A1), in which bending of a material causes signal generation that can be used to determine when the structure passes a certain anatomical feature of interest. Another type of detector that may be employed for this purpose is an EKG detector, which uses the distinct EKG signatures associated with anatomical transition points, such as the entry to the coronary sinus, to determine location of the catheter in the vessel and aid in placement at the desired location.

Additional detectors may be employed, where desired, to provide data which can be employed to modulate operating parameters of the device during aspiration, e.g., aspiration rates, etc. For example, EKG activity may be detected to obtain reference time-points as the flow in the target site may fluctuate as various time points during the EKG cycle; this is especially useful for (1) timing the action of the injection according to EKG patterns, and (2) adapting and modulating aspiration rates to the instantaneous flow inside the target site. In addition, pressure detectors, e.g., for assessment of vacuum efficiency or for detecting CS-pressure signature during device insertion, may be employed, e.g., to provide data which may be used to modulate evacuation rates. Likewise, flow detectors for assessment of baseline flow rate may also be employed for similar purposes. Additional detectors that can be employed include temperature detectors, agent detectors, chemical detectors, etc.

In certain embodiments, the pressure of the target site and/or efferent fluid collection site (which may or may not be the same locations, as described above) and or the tributaries thereof, including a subset of the tributaries thereof, may be modulated, e.g., reduced, in order to achieve the desired collection of agent from the host. The manner in which the pressure may be modulated may vary depending on the particular device employed and manner in which it is implemented, where representative devices and protocols capable of pressure modulation of the target/efferent fluid collection site are described in greater detail below. By modulating the pressure in this manner, one can reduce the pressure within the collection site sufficiently to improve the efficacy of removing the desired agent without causing collapse of the tributaries of the efferent fluid collection site, resulting in a better favorable outcome of the methods.

In certain embodiments, devices that include a shunting element, be it a passive or active shunting element, are employed in a manner that modulates the pressure of the target site and/or efferent fluid collection site, as desired. Alternatively and/or in addition thereto, one can use a pressure sensor within the fluid collection site. The output from such a sensor may be used to optimize the maintenance of the pressure in the collection site so that it is reduced sufficiently in order to increase the likelihood of higher flow to that region from those tributaries that have alternative paths, without causing the collapse of such tributaries.

In certain embodiments, an extension of an aspiration lumen of the device employed is extended selectively into one or more tributaries in order to prevent their collapse during aspiration and to extend the volume from which fluid is aspirated. Alternatively, rather than using a lumen to structurally support the tributaries, a temporary or permanent stent could be introduced to those tributaries prior to aspiration.

In certain embodiments, a specific pattern of aspiration rates that compensates for the delay time between the detection of the desired agent and the activation of the aspiration mechanism is employed. For example, in certain embodiments, there will be a small but finite delay in time between when the agent enters the fluid collection site and when the aspiration mechanism begins to aspirate fluid from the site. During this time delay, some of the fluid containing the agent may have already passed the region (e.g., flowed downstream) from which aspiration occurs at the distal portion of the aspiration lumen, thus potentially reducing the efficacy of retrieving the agent. However, by having a higher rate of aspiration for the early portion of the period in which aspiration occurs, as compared to a rate that more closely resembles the normal physiologic rate of flow within the collection site, e.g., where the higher aspiration rate is 2-fold greater, such as 5-fold or 10-fold greater or more, one can cause that fluid which has already passed the region from aspiration to change direction and return to the aspiration ports. In such cases, the flow modulator can aid in slowing fluid flow at downstream locations from communication port. Once this initial period of a higher rate of aspiration has expired, the aspiration rate could then occur at a lower rate which more closely approximates the normal physiologic rate of flow within the collection site, as desired. Varying aspiration rates may be required at sites where inflow from tributaries follows a cyclical patterns. One example is the inflow from the middle cardiac vein into the coronary sinus, where peaks are registered during systole, and lows during diastole. For this purpose, EKG triggering of aspiration may be employed.

In some embodiments, more than one kind of detector is employed to determine the aspiration parameters and time period. For example, in order to ensure that the leading edge of the agent is successfully aspirated, the activation of the aspiration mechanism may be activated by a counter that counts a conservative, pre-selected number of QRS complexes on an EKG after the beginning of injection of the agent, while the trigger to deactivate the aspiration mechanism may be derived from an optical sensor that can recognize when there is no longer any more agent within the fluid being aspirated. Alternatively, inputs from more than one detector can be used in direct combination with each other to determine the aspiration parameters. For example, due to cardiac motion in the region of a fiber optic based sensor, and/or variations in the rate of flow of the fluid in the region of the sensor, the signal produced may vary in a pattern that is reflective of the cardiac cycle, regardless of whether or not the agent to be detected is present, thus producing a noisy signal. In such a case, the fidelity of the sensor may be augmented by using a filtering algorithm that uses the input from an EKG signal to filter the signal produced by the optical detector. By compensating for changes to the output of the optical detector that are due to the cardiac cycle, it may be easier to more accurately characterize the concentration of the agent to be removed in the region of the detector. Any of the detectors mentioned below may be suitably used in combination with each other to further optimize the detection process and/or the efficacy of the aspiration controller. In certain embodiments, a feedback from the agent injection system can be incorporated into the signal processing algorithm, which ultimately leads to commencement of aspiration.

Practice of the subject methods results in introduction of and selective removal of an agent from a subject (also referred to herein as patient or host), where the amount of agent removed is, in certain embodiments, a substantial portion of (but not all of in certain embodiments) the agent that is introduced in the subject, as described above. Agent is removed by removing fluid, e.g., blood, which contains the agent.

In certain embodiments, the fluid that is removed from the subject or patient may be treated extra-corporeally, e.g., to remove, separate, or neutralize the agent, and then reintroduced into the subject, e.g., where it is desired to minimize the ultimate or final volume of fluid, e.g., blood, that is removed from the subject in a given procedure. For example, where the fluid removed from the subject is blood, the removed blood may be processed with a blood filtering device to separate and remove the agent from the blood, and the processed blood (e.g., filtered fluid), or at least a component thereof (such as red blood cells) be returned to the patient. Examples of fluid, e.g., blood, processing devices include, but are not limited to: the Cell Saver® device (available from Haemonetics); autoLog (available from Medtronic); membrane-based systems, adsorbant-based systems and the like.

As such, the subject methods may include a step of transferring the harvested fluid into a recirculating system to be reintroduced into the body (as described in U.S. Pat. No. 5,925,016, the disclosure of which is herein incorporated by reference). The recirculating system may incorporate mechanisms to separate the substantially undesirable components (e.g., agent) from the substantially desirable components (e.g., removed fluid), such that filtered fluid is produced. Such a system may incorporate a filter, a centrifugal separator, flow cytometry, apheresis or other similar apparatuses. The aspiration mechanism may incorporate fluid characterization elements by which aspirated fluid may be characterized, either quantitatively or qualitatively.

Accordingly, in certain embodiments the subject may be one in which it is desired to keep blood loss at a minimum, e.g., the patient may suffer from coronary artery disease, chronic anemia, etc. Extracorporeal processing and subsequent reinfusion of the treated fluid allows for the reintroduction of the desirable components as an autologous transfusion. Centrifugal mechanisms, filter-based systems, membrane-based systems, adsorbant-based systems, and cell-washing mechanisms are examples of some functional components that can be employed for this purpose.

### Systems

Also provided are systems for use in practicing the subject methods, where the systems include an agent delivery device, an aspiration device for selectively removing agent from the efferent fluid collection site, such as the representative devices described above, and may optionally include one or more additional components that find use in practicing the subject methods, e.g., detectors, processors, data recorders, extracorporeal fluid filtering devices, etc. For example, in some embodiments, a processor can be configured to record data (e.g., hemodynamic/vital parameters at a site), or calculate parameters of interest (e.g., an agent delivery efficiency value) and output the data or calculated parameters, e.g., to a user, or to a display unit, etc. In certain embodiments, the system includes an aspiration controller and aspiration mechanism operatively linked to an aspiration lumen which is introduced into the subject (body), as well as a number of additional/optional components, such as an injection/delivery system for introducing agent into the body at a site upstream of the target efferent fluid collection site, one or more detector elements for detecting the presence of agent in the efferent fluid collection site, and an aspiration recorder/display element for recording data (e.g., fluid flow data, etc.) and displaying the same to the operator. Of interest are the systems described in published United States Application Nos. 20050124969 and 20040254523, the disclosures of which systems described therein modified to include aspiration devices of the present invention are herein incorporated by reference.

In certain embodiments, the injection elements and aspiration elements of the system are linked so that they may be operated in a coordinated fashion, e.g., to minimize agent loss, as described above. For example, in some embodiments, the system can have an agent delivery and removal module configured to automatically modulate or control agent delivery from an agent delivery device (e.g., to a vascular location upstream of a vascular physiological site) and automatically modulate or control aspiration device activation, in a manner sufficient to minimize agent loss. Accordingly, the agent delivery and removal module is one that is configured to automatically adjust agent delivery and aspiration device activation in a manner sufficient to minimize agent loss.

In other embodiments, an agent delivery and removal module can be configured to determine hemodynamic/vital parameters at a site of introduction of agent, and introduce agent at a rate selected in view of the determined hemodynamic/vital parameters. In another embodiment, an agent delivery and removal module can be configured to determine agent introduction parameters at a site of introduction of agent, and activate an aspiration device in a manner based on the determined agent introduction parameters. The agent delivery and removal modules of systems of the invention may be configured to perform one or more of the specified tasks above. The an agent delivery and removal modules may be implemented as any convenient combination of hardware and/or software elements, including circuitry, processors, programming, etc., as desired.

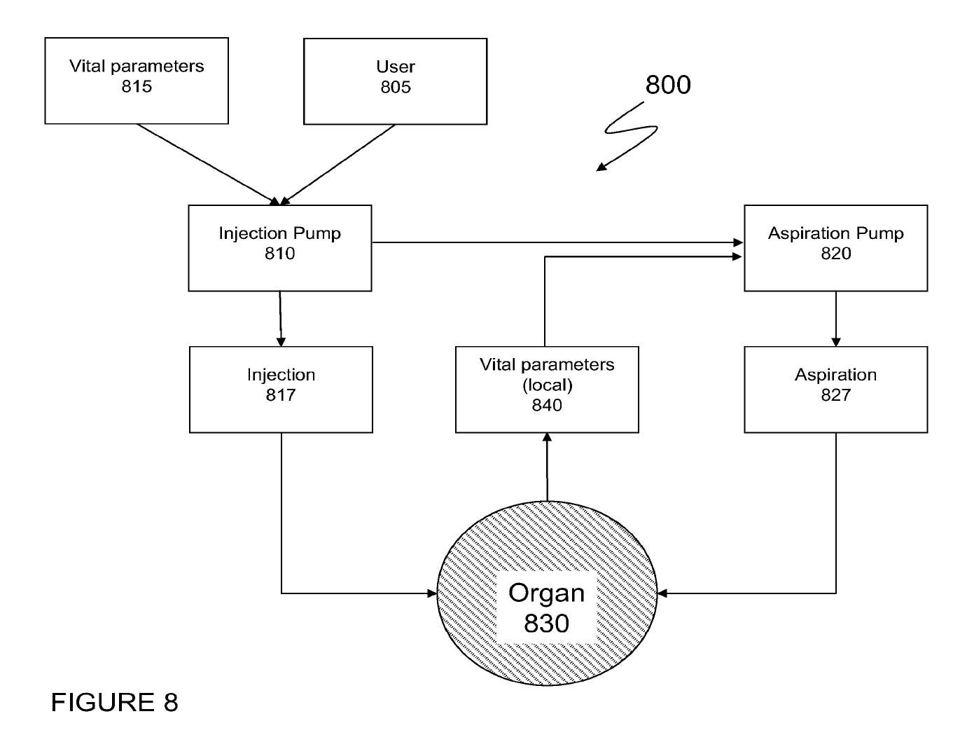

A schematic of a system 800 according to an embodiment of the invention and methods for its use is provided in FIG. 8. In FIG. 8, injection 817 occurs by user activation of the injection pump 805 following the determination of general hemodynamic/vital parameters 815 of the patient such as EKG, blood pressure, etc. In the system shown in FIG. 8, injection data is forwarded from the injection pump 805 to the aspiration pump 820, which activates aspiration 827 of fluid containing agent from the organ 830 of interest in response to the injection data and vital parameters 840 of the organ, if desired. In these embodiments, the method includes activating the aspiration device in a manner based on determined agent introduction parameters (i.e., injection data). As such, the injection system (e.g., pump 810) communicates injection parameters and timing to the aspiration system (e.g., pump 820), which allows for synchronization of aspiration with injection.

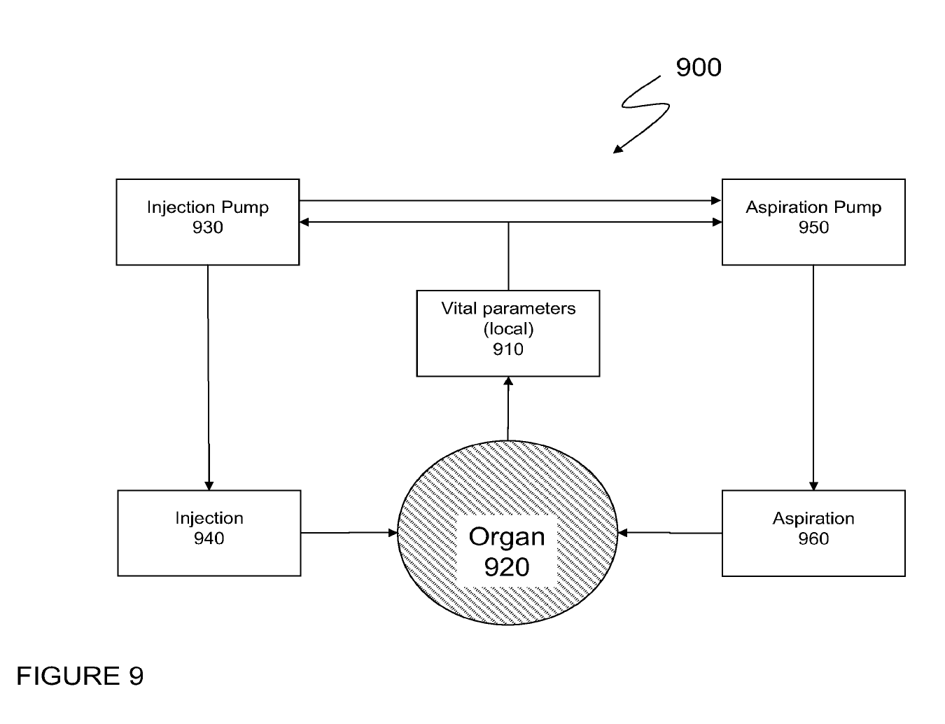

FIG. 9 provides a schematic of a system 900 which is a variation of the system 800 shown in FIG. 8. In FIG. 9, system 900 does not employ general hemodynamic/vital parameters of the patient. Instead, only local hemodynamic/vital parameters 910 collected from within target organ/vessel 920 are employed. Otherwise, injection pump 930 forwards injection data about injection 940 to aspiration pump 950 so that aspiration 960 is coordinated with injection 940 in order to minimize agent loss.

In the systems shown in FIGS. 8 and 9, an agent delivery and removal module (e.g., as described above) may conveniently be employed to coordinate the injection and aspiration in a desired manner, e.g., to minimize agent loss.

### Utility

The subject invention finds use in a wide variety of different applications, including both diagnostic and therapeutic applications. Of interest is the use of the subject methods and devices to selectively introduce agent to and remove agent from a subject, e.g., a locally administered diagnostic or therapeutic agent, so that the host or subject is not systemically exposed to the diagnostic or therapeutic agent.

In certain embodiments, the subject methods are employed to selectively remove a locally administered diagnostic agent, such that the diagnostic agent is only contacted with a limited region or portion of the host to which it is administered, e.g., a specific organ or portion thereof. Vascular physiological sites where the subject methods find use include any suitable site in a body including but not limited to sites in the heart, brain, kidney, lymphatic system, lungs, liver, gastrointestinal system, etc. As such, methods of the invention may be viewed as isolating an organ and then delivering agent thereto such that the agent is locally administered to the organ. A common example of such a compound is a contrast agent, such as a radio-opaque dye. Iodinated forms of such a dye are used routinely during catheter-based interventional procedures such as coronary, renal, neurological, angioplasty, and peripheral arteriography. The iodine component has a high absorption of x-rays and therefore provides a contrast medium for the radiological identification of vessels when introduced within an upstream artery. However, the use of such dyes is known to have potential toxic effects depending on the specific formulation, including direct injury to renal tubule cells, endothelial injury, bronchospasm, inflammatory reactions, pro-coagulation, anti-coagulation, vasodilation and thyrotoxicosis. Other diagnostic agents that can be used with the subject methods include any suitable contrast agent that can be used in a diagnostic procedure, such as with x-rays or other imaging modalities including computed tomography, magnetic resonance imaging, positron emission tomography, nuclear medicine imaging, positron-emission tomography, ultrasound, etc.

Another application of the subject invention is in the selective removal from a patient of a locally administered therapeutic agent, where representative therapeutic agents or materials that may be introduced locally for desired effects but whose direct or other effects would be undesired elsewhere include vasoactive agents, cytotoxic agents, genetic vectors, apoptotic agents, anoxic agents (including saline), photodynamic agents, emboli-promoting particles or coils, antibodies, cytokines, immunologically targeted agents and hormones. Additional agents of interest include, but are not limited to: cells, enzymes, activators, inhibitors and their precursors, as well as sclerosing agents, anti-inflammatories, pro-inflammatories, steroids and osmotic agents, and the like. As such, another representative application of the subject methods is to determine the amount of agent retained at a local area or region of a subject upon local administration of the agent to the subject. For example, where a therapeutic agent is locally administered to a region or location of a subject, e.g., an organ, and blood carrying the agent is selectively removed from the subject according to the subject methods, the amount of agent in the collected blood can be used to determine the amount of agent that was retained by the local region or area, e.g., organ, of the subject. As such, in those cases where the present invention is used to retrieve a diagnostic or therapeutic agent for which a portion of that agent desirably resides in the region into which it is delivered, and the portion of the agent collected from the collection represents an amount of the agent that did not remain resident in that region, the subject methods may be employed to estimate the effective dosage of the agent. For example, in the localized delivery of a chemotherapeutic agent via the afferent branches of a targeted tumor, the present invention is capable of collecting some of the chemotherapeutic agent after it passes through tumor bed, but before it is able to enter into the systemic circulation, thus minimizing its side effects. The difference between the amount of agent injected and the amount of agent that is retrieved by the present invention represents the sum of the amount of agent that was successfully incorporated into the tumor and the amount of agent that escaped to the systemic circulation. If a goal of the localized delivery of the chemotherapeutic agent is to attempt to incorporate a given dosage of the agent into the tumor, it is possible to use the present invention to better estimate how much of the delivered agent was successfully incorporated into the tumor by estimating how much of the agent was retrieved in the collection site. Alternatively, the present invention can be used to allow higher dosage application of agent, the majority of which can then be detected and removed at efferent collection site of the target organ. If a higher than expected amount of agent was retrieved in the collection site, than a substantial portion of the agent was not successfully incorporated into the tumor and this may direct the physician to deliver more agent to the tumor, or consider alternative strategies for treatment. The higher the efficacy of the present invention is in terms of retrieving the agent, the more accurate the estimate of the amount of agent successfully delivered to the site will become.

### Kits

Also provided are kits for use in practicing the subject methods, where the kits may include one or more of the above devices, and/or components of the subject systems, as described above. As such, a kit may include a device, such as a catheter device, that includes an aspiration lumen, aspiration mechanism and aspiration mechanism controller, an injection catheter, etc., as described above. The kit may further include other components, e.g., guidewires, etc., which may find use in practicing the subject methods. For example, a kit for delivering an agent to and removing an agent from a vascular physiological site of a subject can include an agent delivery device comprising a proximal end and a distal end, where the distal end comprises an agent delivery focusing element, and an aspiration device.

In addition to above mentioned components, the subject kits can further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A method for delivering an agent to and removing an agent from a vascular physiological site of a living subject, the method comprising:
(a) positioning:
(i) a distal end of an agent delivery device at a vascular location upstream of the vascular physiological site; and
(ii) an aspiration device at least proximal to the vascular physiological site; and
(b) introducing the agent via the agent delivery device to the vascular location upstream of the vascular physiological site and activating the aspiration device when the agent is at least predicted to be present in the vascular physiological site to remove fluid comprising the agent from the vascular physiological site;
wherein both the introducing of the agent and activating the aspiration device are performed in a manner sufficient to minimize agent loss.

2. The method according to claim 1, wherein the introducing comprises:
determining hemodynamic/vital parameters at the vascular location upstream of the vascular physiological site; and
introducing the agent at a rate selected in view of the determined hemodynamic/vital parameters.

3. The method according to claim 1, wherein the agent delivery device includes an agent delivery focusing element.

4. The method according to claim 3, wherein the agent delivery focusing element is a non-occlusive element.

5. The method according to claim 4, wherein the non-occlusive element comprises a flow modulator.

6. The method according to claim 3, wherein the agent delivery focusing element is an occlusive element.

7. The method according to claim 1, wherein the agent delivery device includes a sensor.

8. The method according to claim 7, wherein the sensor is selected from the group consisting of a flow sensor and a pressure sensor.

9. The method according to claim 1, wherein the method comprises activating the aspiration device in a manner based on determined agent introduction parameters.

10. The method according to claim 1, wherein the aspiration device comprises a flow modulator.

11. The method according to claim 1, wherein the method further comprises determining an agent delivery efficiency value and outputting the determined agent delivery efficiency value to a user.

12. The method according to claim 11, wherein the agent delivery efficiency value is determined by comparing the quantity of agent introduced to the site and the amount of agent removed from the site.

13. The method according to claim 11, wherein the method is performed using a system comprising the agent delivery device, the aspiration device and an agent delivery and removal module configured to automatically adjust agent delivery and aspiration device activation.

14. The method according to claim 1, wherein the agent is a therapeutic agent.

15. The method according to claim 1, wherein the agent is a diagnostic agent.

16. The method according to claim 15, wherein the diagnostic agent is a contrast agent.

17. The method according to claim 1, wherein the vascular physiological site is a cardiac site.

18. The method according to claim 17, wherein the cardiac site is the coronary sinus.

19-20. (canceled)

21. A system for delivering an agent to and removing an agent from a vascular physiological site of a living subject, the system comprising:
an agent delivery device;
an aspiration device; and
an agent delivery and removal module configured to automatically adjust agent delivery and aspiration device activation in a manner sufficient to minimize agent loss.

22-37. (canceled)

38. An agent delivery device for introducing an agent to a vascular physiological site, the device comprising:
a proximal end in fluid communication with a source of the agent; and
a distal end;
wherein the distal end comprises an agent delivery focusing element.

39-44. (canceled)
